(19) United States
(12) Patent Application Publication
(10) Pub. No.: US 2004/0031486 A1
(43) Pub. Date: Feb. 19, 2004

(54) Apparatus for determining physical and/or chemical parameters of aerosol formulations from metered dose inhaler cartridges

(75) Inventors: Thomas Schwarze, Ahlten (DE); Heinz-Joachim Belt, Burgwedel (DE); Michael Pittroff, Hannover (DE); Uwe Becker, Garbsen (DE)

Correspondence Address: CROWELL & MORING LLP, INTELLECTUAL PROPERTY GROUP, P.O. BOX 14300, WASHINGTON, DC, 20044-4300, US

(73) Assignee: Solvay Fluor und Derivate GmbH, Hannover (DE)

(21) Appl. No.: 10/386,947

(22) Filed: Mar. 13, 2003

(30) Foreign Application Priority Data
Mar. 13, 2002 (DE) 202 03 999.4

(51) Int. Cl. A61M 11/00

(52) U.S. Cl. 128/200.23

(57) ABSTRACT

An apparatus for determining the physical and/or chemical parameters of an aerosol formulation from a metered dose inhaler, including a housing (1) with a measuring chamber (8) therein, a multiple sampling head (3) having at least one valve, an opening for receiving the measuring electrode (2), and a mechanism for opening a metered dose inhaler cartridge (5) which includes a sleeve (4), connected detachably or non-detachably to the housing (1), for receiving the metered dose inhaler cartridge, a pin or a needle (7) attached to the surface facing the measuring chamber (8) and protruding into the sleeve (4) for opening the cartridge (5), an actuating element (6) for pressing the cartridge (5) against the pin or needle (7), seals for preventing undesired pressure equalization, and at least one connecting opening (7, 10) to the measuring chamber (8) through which the aerosol formulation can pass unchanged into the measuring chamber.

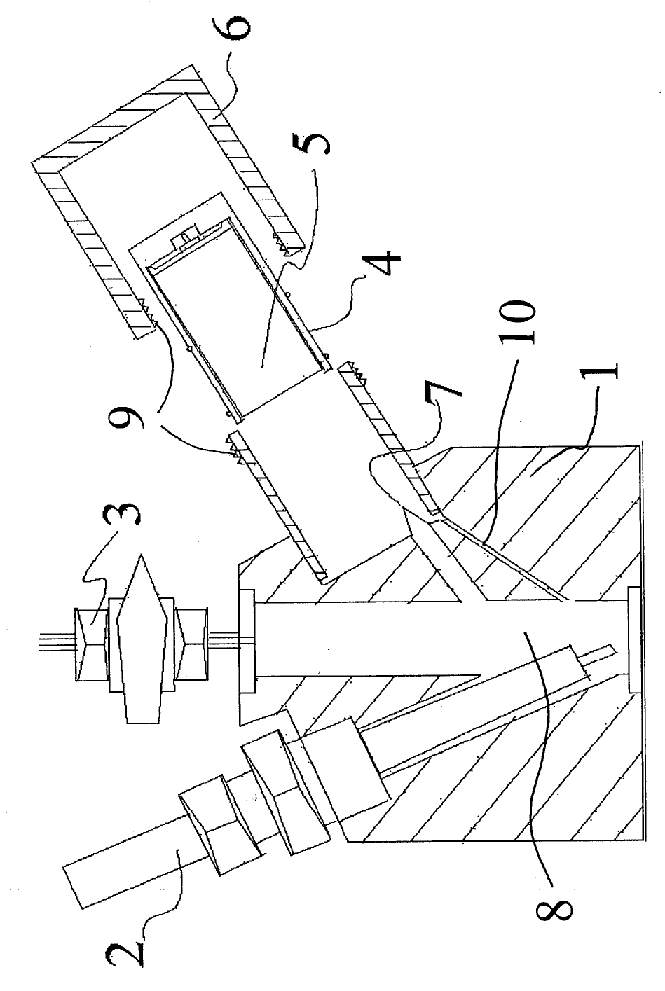

### BACKGROUND OF THE INVENTION

The invention relates to an apparatus for determining the physical and/or chemical parameters of aerosol formulations from metered dose inhalers (MDI).

Metered dose inhalers (also referred to as metering aerosols) have been shown to prolong life by supplying medicaments to patients suffering from asthma, chronic bronchitis or pulmonary emphysema. The handy metered dose inhalers, which are simple to use, serve to deliver a precisely calculated dose of an airway-opening medicament directly into the lungs in acute situations. The release of an active substance by means of a metered dose inhaler is, however, not limited to the aforementioned indications, but is being continually extended, as is known from research reports. These metered dose inhalers comprise a metal cartridge, usually an aluminium container, with a metering chamber and valve system and a holder with a mouthpiece.

In order that a constant, accurately defined release of the active substances can be assured from the first spray burst to the last, propellants are necessary, which release the active substances under constant pressure as a finely divided spray, i.e., the aerosol.

In addition to the pharmaceutical active substance, the complete aerosol formulations also contain, solubilizing agents, surfactants, and fluorinated hydrocarbons, preferably HFC227ea, HFC134a or mixtures thereof, as propellants.

To assure the generation of metered aerosol doses and the quality of such metered doses, it is necessary to determine physical and chemical parameters of the complete aerosol formulation.

### SUMMARY OF THE INVENTION

It is therefore an object of the invention to provide an apparatus for determining the physical and/or chemical parameters of aerosol formulations from metered dose inhalers.

Another object of the invention is to provide an apparatus with which it is possible to introduce a pressurized aerosol formulation without losses into a measuring chamber.

A further object of the invention is to provide an apparatus having a measuring chamber in which the physical and chemical parameters of an aerosol can be determined directly by probes extended into the chamber.

It is also an object of the invention to provide an apparatus with which It is possible to remove a partial amount for further investigation, and which facilitates an online evaluation of the measured results.

These and other objects are achieved in accordance with the present invention by providing an apparatus for determining physical and chemical parameters of an aerosol formulation from a metered dose inhaler, said apparatus comprising a housing with a measuring chamber therein, an opening in said housing for receiving a measuring electrode, a measuring electrode extended through said opening into said measuring chamber, and a metered dose inhaler cartridge opening mechanism. The cartridge opening mechanism releases a metered dose of an aerosol formulation into the apparatus, and means are provided for conveying the released aerosol formulation to the measuring chamber.

A preferred embodiment of the apparatus according to the invention comprises:

a housing (1)

a measuring electrode (2)

a multiple sampling head (3), optionally with valve and/or internal pressure compensation device

a measuring chamber (8) and

a metering-aerosol opening mechanism.

A stirrer (e.g. a magnetic stirrer) may be provided, if desired, on the bottom of the measuring chamber. The measuring chamber may optionally be provided with a discharge connection for evacuating the chamber. If desired, the measuring chamber also may optionally be provided with a thermostat and heating/cooling means to control the temperature.

The metering-aerosol opening mechanism preferably may comprise:

a sleeve (4) for receiving the metal cartridge (5) and means for assuring a connection to the measuring chamber (8). The sleeve 4 may be connected detachably or non-detachably to the housing;

a pin (7) or a needle (7) for opening the cartridge, and

an actuating element (6), preferably a screw-on top, for pressing the cartridge against the needle or pin.

The cartridge (5) is pressed against the pin or needle (7) by the screw-on top (6) and is thus opened to release a dose of the complete aerosol formulation. The aerosol formulation passes through an opening associated with the needle (7) into the measuring chamber. If desired, a further connection or opening (10) between the sleeve and measuring chamber may be provided adjacent the pin or needle to ensure that the aerosol passes into the measuring chamber without loss.

Appropriate seals for avoiding an undesired pressure equalization are also provided.

A measuring electrode (2) extends into the measuring chamber, e.g. for determining the pH value. The measuring electrode may for example also be a conductivity-measuring electrode, a moisture-measuring electrode or an ion-sensitive electrode.

The measuring chamber is made from chemical-resistant material, e.g. stainless steel. It is likewise within the scope of the invention to provide the measuring chamber with a viewing window.

The apparatus of the invention makes it possible to transfer the contents of the cartridge into the measuring chamber without the composition of the metered aerosol formulation being changed.

It is likewise within the scope of the invention to provide the apparatus of the invention with removal openings, e.g. valves, capillaries etc., arranged to communicate with the measuring chamber, so that partial samples of the gas or liquid phase of the aerosol formulation contained in the measuring chamber can be removed for further investigation or analysis in external measuring apparatus.

Suitable external measuring apparatus with which the apparatus of the invention may be used include, for example, gas chromatographs (GC), hygrometers, X-ray spectrometers, mass spectrometers, polarimeters, crystal structure analyzers, calorimeters, refractometers, high pressure liquid chromatography (HPLC) apparatus, nuclear magnetic resonance (NMR) apparatus, electrophoresis apparatus, apparatus for measuring the TIC and TOC contents, apparatus for recording adsorption in the infrared (IR), ultraviolet (UV) or Fourier transform infrared (FTIR) range, or measuring arrangements for determining the solubility, the permeation behavior, the thermal conductivity, and/or the contents of individual constituents (e.g. by means of AAS), and also combinations of such devices (e.g. GC-MS, GC-IR, GC-FTIR).

It is also possible within the scope of the invention to connect means for online measured data acquisition to the apparatus of the invention. This is particularly advantageous in controlling manufacturing processes for producing the aerosol formulation.

### BRIEF DESCRIPTION OF THE DRAWING

The invention will be described in further detail hereinafter with reference to an illustrative preferred embodiment shown in the accompanying drawing which is a schematic view of a simplified embodiment of the apparatus of the invention.

### DETAILED DESCRIPTION OF PREFERRED EMBODIMENT

The drawing FIGURE schematically depicts an apparatus comprising a housing 1 having a measuring chamber 8 therein. A measuring electrode 2 is extended through an opening in the side of the housing 1 into the measuring chamber 8. At the end of the measuring chamber 8 there is a multiple sampling head 3, optionally containing a valve. Connected to one side of the housing 1 is a sleeve 4 for receiving a metal aerosol cartridge 5 of a metered dose inhaler. An actuating element 6 provided with a thread 9 serves to urge the cartridge 5 against a pin or hollow needle 7 at one end adjacent the measuring chamber 8. This causes the cartridge 5 to release a measured dose of the aerosol into the sleeve 4, from whence it passes through a hollow in needle 7 and/or through a connecting passage 10 into measuring chamber 8 for analysis by measuring electrode 2.

The foregoing description and example have been set forth merely to illustrate the invention and are not intended to be limiting. Since modifications of the described embodiment incorporating the spirit and substance of the invention may occur to persons skilled in the art, the invention should be construed broadly to include all variations within the scope of the appended claims and equivalents thereof.

What is claimed is:

1. An apparatus for determining physical and chemical parameters of an aerosol formulation from a metered dose inhaler, said apparatus comprising a housing with a measuring chamber therein, an opening in said housing for receiving a measuring electrode, a measuring electrode extended through said opening into said measuring chamber, and a metered dose inhaler cartridge opening mechanism.

2. An apparatus according to claim 1, further comprising a multiple sampling head having at least one valve communicating with said measuring chamber.

3. An apparatus according to claim 1, wherein the measuring chamber is provided with at least one pressure relief valve.

4. An apparatus according to claim 1, wherein the measuring electrode is a pH-measuring electrode, a conductivity-measuring electrode, a moisture-measuring electrode or an ion-sensitive electrode.

5. An apparatus according to claim 1, wherein the metered dose inhaler cartridge opening mechanism comprises a sleeve connected to the housing for receiving a cartridge of the metered dose inhaler, a protruding pin or needle attached to a surface of said sleeve adjacent said measuring chamber for opening the cartridge, an actuator for pressing the cartridge against the pin or needle, seals for preventing undesired pressure equalization, and at least one connecting opening leading from the sleeve to the measuring chamber through which the aerosol formulation can enter the measuring chamber.

6. An apparatus according to claim 5, wherein said sleeve is detachably connected to said housing.

7. An apparatus according to claim 5, wherein said sleeve is permanently fixed to said housing.

8. An apparatus according to claim 1, wherein the measuring chamber is connected to at least one further measuring device.
